(19) United States
(12) Patent Application Publication
(10) Pub. No.: US 2026/0083337 A1
(43) Pub. Date: Mar. 26, 2026

(54) METHODS FOR MEASURING BLOOD PRESSURE USING A WEARABLE DEVICE

(71) Applicant: Wear It System Holding S.A., Plan- les- Ouates (CH)

(72) Inventors: Iddo Johanan Gideon Hanse, Plan- les- Ouates (CH); Kambiz Ebrahimkheil, Plan- les- Ouates (CH)

(21) Appl. No.: 19/390,345

(22) Filed: Nov. 14, 2025

Related U.S. Application Data
(62) Division of application No. 18/809,752, filed on Aug. 20, 2024.

(30) Foreign Application Priority Data
Aug. 23, 2023 (NL) 2035661

(51) Int. Cl. A61B 5/021; A61B 5/00; A61B 5/024

(52) U.S. Cl. CPC A61B 5/02116; A61B 5/02108; A61B 5/6801; A61B 5/7203; A61B 5/7221; A61B 5/7225; A61B 5/7235; A61B 5/7264; A61B 5/02416

(57) ABSTRACT

Methods for non-invasively measuring blood pressure using multi-wavelength photoplethysmography (PPG) signals obtained from peripheral blood vessels. A wearable device is calibrated using PPG signals measured distal to the wearable device and analyzed in order to create a cardiovascular profile of a wearer. After calibration, windows of PPG signals are analyzed to provide a blood pressure estimation, based on the created profile. The current invention utilizes single-site measured PPG signals in order to make a prediction of the wearer's blood pressure. By using a combination of multi-wavelength PPG waveform features and a range of vital signs calculated by the wearable device, the device is able to provide a more accurate and robust prediction of blood pressure compared to existing methods. This has important implications for clinical settings where frequent and remote monitoring of blood pressure is necessary for managing a range of health conditions.

### CROSS-REFERENCE TO RELATED APPLICATIONS

This application is a divisional of and claims priority to and the benefit of U.S. patent application No. Ser. No. 18/809,752, entitled “Wearable Device for Measuring a Blood Pressure of a Wearer”, filed Aug. 20, 2024, which claims priority to and the benefit of Netherlands Patent Application No. 2035661, entitled “A WEARABLE DEVICE FOR MEASURING A BLOOD PRESSURE OF A WEARER”, filed Aug. 23, 2023, and the specifications and claims thereof are incorporated herein by reference.

### BACKGROUND OF THE INVENTION

### Field of the Invention

The invention relates to methods for non-invasively measuring blood pressure using multi-wavelength photoplethysmography (PPG) signals obtained by a wearable device from peripheral blood vessels.

### Background

Photoplethysmography (PPG) is a non-invasive optical technique that measures blood volume changes in the microvascular bed of tissue. It works by shining a light source, typically an LED, onto the skin and detecting the amount of light that is transmitted or reflected back to a photodetector. This optical signal can be used to derive information about blood flow, heart rate, and other physiological parameters.

Blood pressure estimation based on photoplethysmography (PPG) signals has received increasing attention in recent years due to the non-invasive and continuous nature of PPG measurements. There are a variety of algorithms that have been proposed for estimating blood pressure from PPG signals, including methods that utilize waveform-based feature extraction, frequency features of the input signal, and combinations of both. Some of these methods also incorporate other physiological signals, such as electrocardiogram (ECG) and PPG sensors from multiple locations on the body, to obtain an indication of Pulse-Transit Time (PPT), a feature highly correlated with blood pressure. Although accurate, having sensors placed on multiple locations of the body imposes new challenges, such as placement determination and an increased chance of noisy signals.

Moajjem Hossain Chowdhurry et al. in their article in Sensors 2020, proposed a method for estimating blood pressure (BP) based on PPG signals wherein time, frequency and time-frequency domain features are extracted from the PPG signals and fed in to ML algorithms to estimate systolic BP and diastolic BP.

US20230082362 and Akuthota Chandra et al. in their publication in IEEE 2022 proposed measuring BP from single PPG.

El-Hajj et al. in their publication in biomedical signal processing and control 2020, proposed using machine learning and PPG signal processing to estimate blood pressure.

The existing single measurement site PPG-based blood pressure algorithms are based on single-wavelength signal, and use time-and frequency domain features in order to estimate blood pressure. These features are often used as input in machine learning methods to train a model that maps the combination of features to the associated systolic and diastolic blood pressure values. A problem with only using time-and frequency domain features based on the direct ppg signal is a high overlap and correlation of features, making the algorithms prone to overfitting on certain characteristics in the PPG signal.

### BRIEF SUMMARY OF THE INVENTION

It is an object of the current invention to correct the short-comings of the prior art and to provide a solution for measuring a blood pressure of a wearer using single-site measurements while providing measurements accuracy better than that of multiple-site measurements.

This and other objects which will become apparent from the following disclosure, are provided with a wearable device having the features of one or more of the appended claims.

In a first aspect of the invention, the wearable device is configured for processing a photoplethysmography signal and for emitting light at a plurality of light-wavelengths for measuring a blood pressure of a wearer of said device, wherein the photoplethysmography signal comprises a plurality of pulses and wherein a pulse is the photoplethysmography signal between two consecutive valleys, wherein, for each light-wavelength, the wearable device is configured for:
creating a photoplethysmography segment by acquiring the photoplethysmography signal within a predefined time window;
collecting at least one biologically vital sign, such as a heart rate of the wearer, a respiration rate of the wearer, and an oxygen saturation rate of the wearer;
collecting at least one waveform-based parameter proportional to a peak amplitude of said photoplethysmography segment;
collecting at least one time-based parameter proportional to a peak time of said photoplethysmography signal or proportional to a peak width of said photoplethysmography segment;
creating a cardiovascular profile vector of the wearer;
calculating a mean squared error between the collected profile vector and a profile vector belonging to previously predicted cluster of profile vectors, not necessarily profile vectors of the current wearer; and
estimating a blood pressure of the wearer using the photoplethysmography segments comprised in the cardiovascular profile of the wearer when said mean squared error is smaller than a first threshold value.

Advantageously, the wearable device is configured for filtering the photoplethysmography segment, preferably by using a bandpass filter, and using said filtered photoplethysmography segments in the remaining steps of the method.

More advantageously, the wearable device is configured for creating a cardiovascular profile vector of the wearer comprises concatenating the collected at least one waveform-based parameter, the collected at least one time-based parameter, the collected at least one biologically vital sign and corresponding non-eliminated photoplethysmography segments into a single profile vector.

Multi-wavelength PPG sensors have the advantage of being able to measure changes in different wavelengths of light, which can provide more information about the hemodynamic changes occurring in tissue. For example, different wavelengths of light can penetrate to different depths in tissue, allowing for measurements of both arterial and venous blood flow. Additionally, different wavelengths of light are absorbed differently by different chromophores, such as oxygenated and deoxygenated hemoglobin, which can provide insights into tissue oxygenation and metabolism. By combining information from multiple wavelengths, multi-wavelength PPG sensors can provide a more comprehensive picture of cardiovascular function and tissue perfusion, which are used to calculate informative features from the PPG signal, to use as input for the blood pressure estimation algorithm. Existing works do not address the inter-wavelength characteristics of PPG signals and their predictive capabilities to estimate blood pressure.

Furthermore, a well-known problem in PPG-based solutions is the capricious nature of the signal, meaning that the waveform of the PPG signal can display sudden changes in characteristics in a relatively short amount of time. This poses a challenge for beat-to-beat prediction in real-life settings. To overcome this problem, the current invention is based on set timeframes of PPG signals, in which a multi-wavelength signal quality indication method isolates only the useful and high-quality signals. These signals are then used to calculate both waveform-based parameters, time-based parameters, and a combination of the two.

Advantageously, the wearable device is optionally configured for normalizing the collected at least one time-based parameter by multiplying said at least one time-based parameter by a factor inversely proportional to a peak width of the photoplethysmography segment. This ensures consistency across different sensors or patients, helping to maintain accuracy when the data is analyzed and compared.

Suitably, the wearable device is configured to emit light comprising light-wavelengths between 400 nm and 1000 nm.

More suitably, the wearable device is configured to emit light comprising:
a light-wavelength of 525 nm;
a light-wavelength of 660 nm; and
a light-wavelength of 880 nm.

In real-world scenarios, not all collected PPG signals are of high quality. Various factors, such as patient movement or sensor placement, may degrade the signal quality, leading to erroneous feature extraction and thus inaccurate blood pressure predictions. To mitigate this, our system employs a Signal Quality Indicator (SQI). Advantageously, for each photoplethysmography segment, the wearable device is preferably configured for:
calculating at least one of the following values for each pulse of said photoplethysmography segment:
   a pulse wave amplitude left by calculating a difference between vertical amplitudes of a first peak and a first valley of the pulse;
   a pulse wave amplitude right by calculating a difference between vertical amplitudes of the first peak and a second valley of the pulse;
   a pulse wave duration by calculating a difference between a time of the second valley and a time of the first valley of the pulse;
   a rise time by calculating a difference between a time of the first peak and a time of the first valley of the pulse;
   a systolic-to-diastolic duration ratio by calculating the ratio between a difference between the rise time and a difference between the time of the second valley and the time of the first peak of the pulse;
eliminating a photoplethysmography pulse when:
   the rise time of said pulse is outside a predefined first range, optionally [0.01-10] second or [0.08-0.49] seconds; or
   the pulse wave duration of said pulse is outside a predefined second range, optionally [0.01-10] second or [0.3-2] seconds; or
   a ratio or an inverse ratio between the pulse wave amplitude right and the pulse wave amplitude left is smaller than a second threshold value e.g. 50%; or
   the systolic-to-diastolic duration ration is larger than a third threshold optionally equal to 5 or 3 or 1.1;
calculating a signal quality index of the photoplethysmography segment by calculating a ratio of non-eliminated photoplethysmography pulses over a total number of photoplethysmography pulses comprised in said photoplethysmography segment.

Furthermore, the wearable device is preferably configured for eliminating the photoplethysmography segment when signal quality index of said photoplethysmography signal is lower than a fourth threshold value. The wearable device is preferably configured for setting said fourth threshold value at 20%. Only PPG segments having an SQI higher than the fourth threshold value (e.g. higher than 20%) are retained for further processing. This quality control step is crucial as it ensures the reliability and accuracy of the subsequent feature extraction and blood pressure prediction stages. By rejecting poor quality signals at an early stage, the system significantly reduces the chances of model contamination and error propagation.

To further adapt the PPG measurements, and subsequently the blood pressure predictions, to the unique physiology of the wearer, the wearable device is preferably configured for:
collecting the wearer demographic information such as age, sex, body weight and height; and
adding said demographic information into the cardiovascular profile of the wearer.

Advantageously, the wearable device is configured for grouping profile vectors into a plurality of clusters based on similarity in at least one of the vital signs and on similarity in the wearer demographic information by a using a silhouette coefficient to determine an optimal number of clusters for establish a K-Means clustering on said profile vectors.

Additionally, the wearable device is preferably configured for training a Random Forest model on each cluster by combining a plurality of decision trees and training each decision tree on a separate subset data of said cluster. In fact, the wearable device is preferably configured for employing a variety of machine learning techniques for model training and calibration. After preprocessing, normalization, and scalarization of the input data, a classification model identifies the appropriate user subgroup based on demographic features and features extracted from the initialization measurements. The predictive model, trained using a piecewise ensemble method, utilizes the cross-validated mean-squared error, or cross-entropy, or a combination thereof as a loss function. This involves splitting the training data into multiple subsets, training the ensemble model on a subset, and testing it on the remaining data. This process is iteratively repeated with different data subsets to robustly evaluate model performance.

The cross-validated mean squared error, for example, is computed by averaging the squared differences between predicted and actual blood pressures across all subsets. This error squaring emphasizes larger errors and guarantees positive values. The cross-validated mean squared error offers a comprehensive performance metric for the model. The trained model is then used to map input features into numerical values that yield the predicted systolic and diastolic blood pressure readings.

Suitably, the wearable device is preferably configured for:
assigning a new wearer to one of the plurality of clusters based on the demographic information of the new wearer and based on at least one of the vital signs of said new wearer; and
using the Random Forest model corresponding to said cluster as a starting point, and fine-tuning said starting point to create a cardiovascular profile for the new wearer using a regularization framework such as a Lasso regression, a Ridge regression or an Elastic Net.

More suitably, the wearable device is preferably configured for updating the cardiovascular profile of the wearer by performing the steps of the preceding claims on new sets of acquired photoplethysmography segments. This is for creating a patient-specific model that represents a fusion of generalized cluster-based learning and personalized fine-tuning.

In a second aspect of the invention, the wearable device configured to emit light onto a skin of a wearer and to receive light emitted from the skin of the wearer wherein the device comprises a computer system capable of performing the steps according to any one of the aforementioned aspects.

Reference to a first “aspect” of an invention is not intended to suggest that such embodiment is a preferred embodiment or best mode. Objects, advantages and novel features, and further scope of applicability of the present invention will be set forth in part in the detailed description to follow, taken in conjunction with the accompanying drawings, and in part will become apparent to those skilled in the art upon examination of the following, or may be learned by practice of the invention. The objects and advantages of the invention may be realized and attained by means of the instrumentalities and combinations particularly pointed out in the appended claims.

### BRIEF DESCRIPTION OF THE SEVERAL VIEWS OF THE DRAWINGS

The accompanying drawings, which are incorporated into and form a part of the specification, illustrate one or more embodiments of the present invention and, together with the description, serve to explain the principles of the invention. The drawings are only for the purpose of illustrating one or more embodiments of the invention and are not to be construed as limiting the invention. In the drawings:

FIG. 1 is a schematic illustration showing various operations of a wearable device according to an embodiment of the present invention;

FIG. 2 is a schematic illustration showing various operations of a wearable device according to an embodiment of the present invention;

FIG. 3 is a schematic illustration showing various operations of a wearable device according to an embodiment of the present invention; and

FIG. 4 shows two graphs related to the operation of a wearable device according to an embodiment of the present invention.

### DETAILED DESCRIPTION OF THE INVENTION

### Data Acquisition and Preprocessing

Embodiments of the present invention are directed to a wearable device that takes in various forms of data such as demographic details (like age, sex, weight, height and skin color), vital signs (including heart rate, respiratory rate and SpO2) and PPG signals. These PPG signals are captured at a plurality of light-wavelengths, in particular at three distinct wavelengths, namely red (660 nm), green (525 nm), and infrared (880 nm). The selection of these specific wavelengths is strategic as they each penetrate the skin at different depths, allowing for the capture of arterial pulsations on multiple levels. This multi-level capture improves the reliability of the signal and reduces the impact of motion artifacts, an important feature for practical usability of the system.

Following the collection of raw PPG signals, the data undergoes a preprocessing phase. During this phase, a bandpass filter such as a Chebyshev filter with a predefined general range of e.g. [0.3-5] Hz is applied to remove the high-frequency noise and baseline wander from the signals. The exact frequencies that are filtered out are determined by the corresponding vital signs to the PPG segment. For example, the respiration rate generally causes a baseline wander between 0.1-0.6 Hz, for which the corresponding frequency is filtered out of the ppg signal. The signals are normalized to a uniform scale. This ensures consistency across different sensors or patients, helping to maintain accuracy when the data is analyzed and compared.

### Signal Quality Assessment

In real-world scenarios, not all collected PPG signals are of high quality. Various factors, such as patient movement or sensor placement, may degrade the signal quality, leading to erroneous feature extraction and thus inaccurate blood pressure predictions. To mitigate this, the method of the current invention employs a Signal Quality Indicator (SQI).

Only PPG segments having a signal quality index higher than the 4^{th} threshold value (e.g. 20%) are retained for further processing. This quality control step is crucial as it ensures the reliability and accuracy of the subsequent feature extraction and blood pressure prediction stages. By rejecting poor quality signals at an early stage, the system significantly reduces the chances of model contamination and error propagation.

### PPG Parameters Extraction

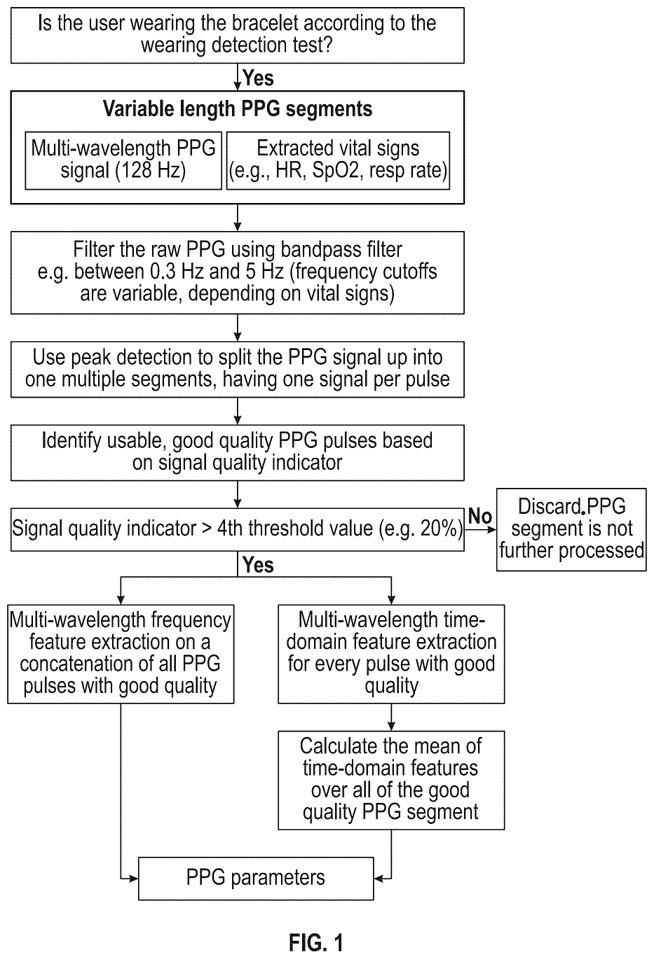

In the post-preprocessing phase, the wearable device of the current invention is optionally configured for extracting time-based parameters and waveform-based parameters from each PPG wavelength signal and a combination of PPG wavelength signals. This includes information like pulse amplitude, peak-to-peak intervals, signal energy, spectral entropy, and frequency peaks from spectral analysis. These features, combined from all three wavelengths, are then concatenated into PPG parameters vectors. This set serves as a robust representation of the wearer's cardiovascular profile, capturing both the temporal and spectral dynamics of the cardiovascular system. An extensive list of waveform-parameter and time-based parameters is given in appendix 1. FIG. 1 illustrates the step of data acquisition and preprocessing, the step of calculating the SQI, and the step extracting PPG parameters.

### Cluster Identification

Once a plurality of wearers'cardiovascular profiles are created, a K-Means clustering algorithm is employed to obtain clusters of ppg segments that contain datapoints that are similar to each other in terms of vital signs and patient demographics. Since the relationship between the characteristics of the PPG signal (features) and the blood pressure is different under different conditions, these clusters are differentiated in order to train the parameters for the prediction model for each cluster.

K-Means is a versatile algorithm, capable of effectively partitioning data into distinct groups based on their similarities. To determine the optimal number of clusters (K), the Silhouette Coefficient is used. This metric provides a measure of how similar an object is to its own cluster compared to other clusters. In this way, the computer-implemented method is able to segregate the wearers population into distinct cardiovascular profiles, improving the homogeneity of the groupings, which is a key factor in the accuracy of the subsequent predictive models.

### Cluster-based Model Training

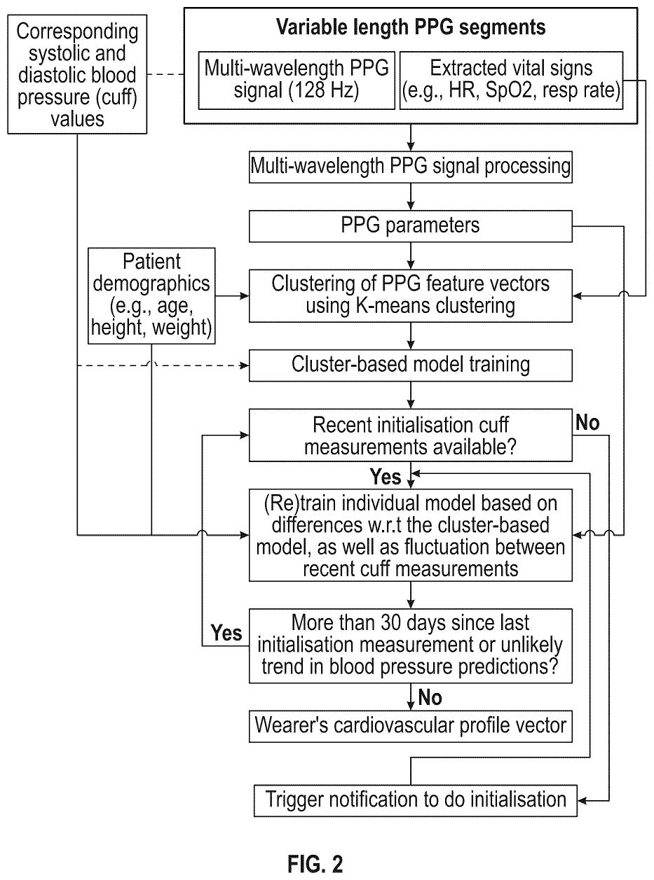

For each identified cluster, the wearable device is configured for training a Random Forest model. This model type is specifically chosen for its ability to handle high-dimensional data and its inherent resistance to overfitting through ensemble learning. It essentially combines multiple decision trees, each trained on a different subset of the data, thereby improving model robustness and predictive accuracy. The most important features differ per cluster, which are automatically extracted by the random forest ensemble technique. This ensures a limited selection of the extensive feature list per cluster model. During training, the wearable device is configured for optimizing model hyperparameters through a technique called grid search, combined with a 5-fold cross-validation setup. This rigorous approach helps balance model complexity with prediction accuracy, a crucial aspect in building effective predictive models. The cluster identification process and the cluster-based training are illustrated in FIG. 2.

### Personalized Model Training

Whenever new wearers are introduced to the database, they are assigned to one of the pre-identified clusters. This assignment is based on their demographic, vital signs, and PPG features. The system then uses the corresponding cluster's Random Forest model as a foundational structure, and fine-tunes it to the wearer's unique physiology. Here, the computer-implemented method comprises the step of using a regularization framework such as a Lasso model, known for its regularization properties that encourage a sparse solution, thus effectively preventing overfitting. In this manner, the method of the current invention comprises the step of creating a wearer-specific model that represents a fusion of generalized cluster-based learning and personalized fine-tuning.

### Personalized Model Updating

The wearable device is configured to continuously learn and adapt. As new calibration data is received from the wearer, which includes concurrent PPG signals and blood pressure measurements, the computer-implemented method comprises the step of performing incremental updates on the patient-specific regularization framework parameters. This enables the model to adapt to the wearer's unique and evolving cardiovascular dynamics, thereby maintaining accuracy over time.

### Prediction

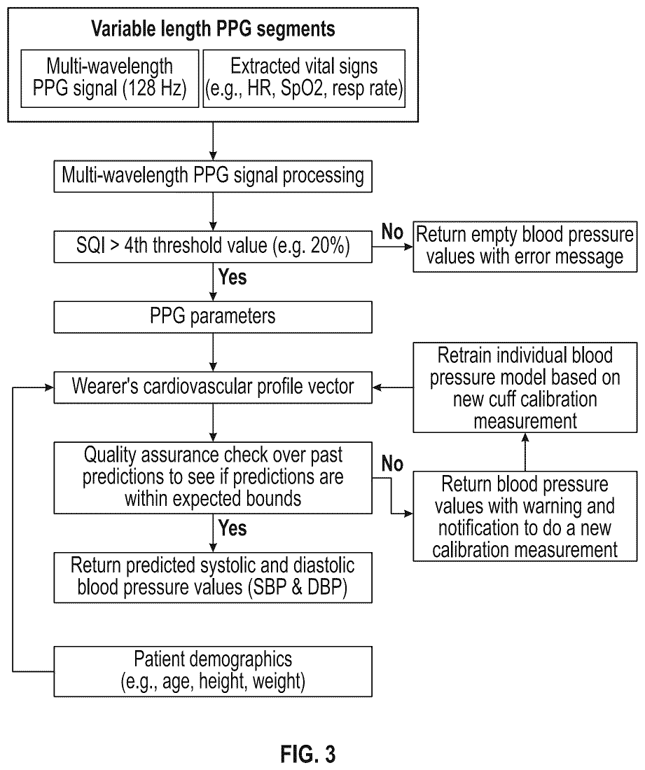

The fine-tuned wearer-specific regularization framework is used by the system to predict blood pressure. In cases where calibration data is scarce or insufficient for comprehensive fine-tuning, the system reverts to the cluster-based Random Forest model, thereby ensuring reliable and consistent predictions at all times. The personalized model training, the personalized model updating and the prediction process are illustrated in FIG. 3.

Different vitals do not affect the PPG signal in itself so much, but rather the interaction between the PPG signal and the blood pressure. The majority of the information is not found in differences in PPG features, but rather in the interaction effect between vital signs and PPG features.

The clustering is therefore not just used for how the signals are filtered, but the more important part is how they are handled in the rest of the algorithm.

For example, the PPG signal and its corresponding feature values at a moment of very high heart rate for a person with low blood pressure might be very similar to the PPG feature values seen in a measurement for a person with high blood pressure that is in a lower heart rate region. As these interactions effects are explained by big differences in vital signs, it is important to classify mentioned PPG segments into different groups of activity level (respiration rate, heart rate, blood oxygen saturation).

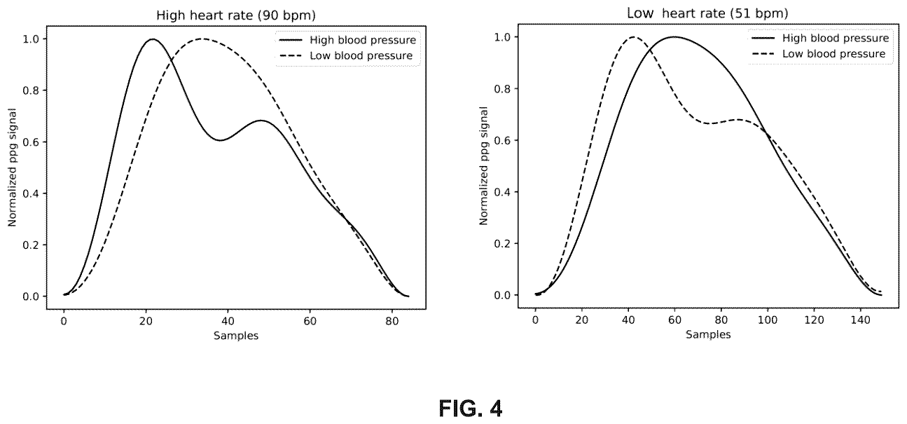

To better understand how heart rate could affect the PPG signal under multiple conditions, FIG. 4 illustrates four PPG curves that have been found in a patient in multiple conditions. As shown in FIG. 4, the PPG signal corresponding to a measurement with high BP and low HR (heartrate) looks very much like the signal corresponding to a measurement with low BP and high HR. In addition to controlling for the difference in absolute length of the signal (+−150 samples in low HR condition vs. +−85 samples in high HR condition) by normalizing the calculated features, there are more interaction effects at play.

The novelty of the wearable device of the current invention lies in the interaction effect between the extracted PPG parameters and blood pressure under different settings of vitality. The wearable device is optionally configured for training multiple models corresponding to distinct combinations of biologically vital signs and/or demographic ranges, thereby accounting for different physiological and lifestyle factors that might influence blood pressure.

Ranges for the biologically vital signs, such as heart rate, respiration rate, and blood saturation can be determined by applying a clustering algorithm to the data of a plurality of wearers. This ensures an adaptive segmentation of vital signs according to the underlying distribution of wearers.

The demographics data, including age, BMI (Body Mass Index), and gender, can also be divided into specific ranges. These are defined according to conventional categories.
BMI: underweight, normal weight, overweight, obese I, obese II, obese II;
Age: (0, 18), (19, 30), (31, 45), (46, 55), (56, 65), (66, 75), (75, 85), (85, 100);
Gender: Male/female.

The wearable device is configured for training individual models for every unique combination of the defined ranges of biologically vital signs and/or demographics. This results in a multi-dimensional matrix of models, allowing for a high degree of specificity in capturing the complex interactions between these variables and blood pressure. The training process comprises the following steps:
a. Filtering Data: The data is filtered according to each specific combination of ranges, resulting in subsets of the data that correspond to each model.
b. Model Training: A predictive model is trained on each subset of the data, with blood pressure as the target variable.
c. Model Saving: Trained models are saved, indexed by the corresponding combination of ranges, for subsequent retrieval during prediction.

The prediction process can handle new PPG data with or without accompanying demographic information. The steps comprise:
a. Identifying Ranges: The corresponding ranges for the vital signs and available demographics are identified using the same clustering or segmentation logic applied during training.
b. Utilizing Default Demographics: If demographic data is not provided, the default values for age, BMI, and gender are calculated by taking the mean range from the training data with ppg data that has the most similar pattern to the current window.
   These values are used when (some of the) demographic information is not available during prediction.
c. Model Loading: The specific model corresponding to the identified combination of vital signs and demographics ranges is loaded.
d. Prediction: The loaded model is used to predict the blood pressure based on the incoming PPG data.

The wearable device of the current invention offers a versatile and highly specific approach to blood pressure prediction, accommodating complex interactions between biologically vital signs, PPG features, and demographic factors. By segmenting the data into ranges defined by clustering and training distinct models for each combination, the method provides personalized and nuanced insights into blood pressure behavior. The ability to handle missing demographic data adds to its adaptability and broad applicability in various medical and healthcare settings. In other words, the method of the current invention combines the strengths of generalized models derived from clustered population data with the advantages of personalized models incrementally fine-tuned on individual calibration data. By harnessing PPG signals of multiple wavelengths and adopting a continuous learning approach, it offers an inclusive, robust solution that is adaptable to diverse patient characteristics and continuously enhances its accuracy as it processes more data.

Although the invention has been discussed in the foregoing with reference to an exemplary embodiment of the wearable device of the invention, the invention is not restricted to this particular embodiment which can be varied in many ways without departing from the invention. The discussed exemplary embodiment shall therefore not be used to construe the appended claims strictly in accordance therewith. On the contrary the embodiment is merely intended to explain the wording of the appended claims without intent to limit the claims to this exemplary embodiment. The scope of protection of the invention shall therefore be construed in accordance with the appended claims only, wherein a possible ambiguity in the wording of the claims shall be resolved using this exemplary embodiment.

Embodiments of the present invention can include every combination of features that are disclosed herein independently from each other. Although the invention has been described in detail with particular reference to the disclosed embodiments, other embodiments can achieve the same results.

Variations and modifications of the present invention will be obvious to those skilled in the art and it is intended to cover in the appended claims all such modifications and equivalents. The entire disclosures of all references, applications, patents, and publications cited above are hereby incorporated by reference. Unless specifically stated as being “essential” above, none of the various components or the interrelationship thereof are essential to the operation of the invention. Rather, desirable results can be achieved by substituting various components and/or reconfiguration of their relationships with one another. The terms, “a”, “an”, “the”, and “said” mean “one or more” unless context explicitly dictates otherwise. The terms “about” or “approximately” as used herein, mean an acceptable error for an articular recited value, which depends in part on how the value is measured or determined. In certain embodiments, “about” can mean one or more standard deviations. When the antecedent term “about” is applied to a recited range or value it denotes an approximation within the deviation in the range or value known or expected in the art from the measurement method. For removal of doubt, it should be understood that any range stated in this written description that does not specifically recite the term “about” before the range or before any value within the stated range inherently includes such term to encompass the approximation within the deviation noted above.

Optionally, embodiments of the present invention can include a general or specific purpose computer or distributed system programmed with computer software implementing steps described above, which computer software may be in any appropriate computer language, including but not limited to C++, FORTRAN, ALGOL, BASIC, Java, Python, Linux, assembly language, microcode, distributed programming languages, etc. The apparatus may also include a plurality of such computers/distributed systems (e.g., connected over the Internet and/or one or more intranets) in a variety of hardware implementations. For example, data processing can be performed by an appropriately programmed microprocessor, computing cloud, Application Specific Integrated Circuit (ASIC), Field Programmable Gate Array (FPGA), or the like, in conjunction with appropriate memory, network, and bus elements. One or more processors and/or microcontrollers can operate via instructions of the computer code and the software is preferably stored on one or more tangible non-transitive memory-storage devices.

### Appendix 1

From the PPG segments, time-based and waveform-based parameters are extracted to be used as input for the machine learning model. To get a valid prediction, a combination of statistical features, frequency domain features, demographic features, first/second derivative features, width related PPG features and features from the PPG signal are used. All time-domain features are scaled by dividing by the length of the ppg-pulse, to eliminate the effect of heartrate on the trained models, as the heartrate is extremely correlated with any time-domain features.

### LIST OF COLLECTED AND EXTRACTED FEATURES

Systolic peak,
Diastolic peak,
Height of Notch,
Systolic peak time (spt),
Diastolic peak time (dpt),
Height of Notch Time (hnt),
Time delta (dt),
Pulse interval (pi)
Peak-to-peak interval (ppi)
Pulse width
Inflection point area
Augmentation index
Alternative augmentation index
Systolic peak output curve
Diastolic peak downward curve
Height of signal at 25% of the systolic peak downward curve (h25_sdt)
Height of signal at 50% of the systolic peak downward curve (h50_sdt)
Height of signal at 75 % of the systolic peak downward curve (h75_sdt)
h25_sdt/h50_sdt
h25_sdt - h50_sdt
(spt)/(ppi)
(hnt)/(ppi)
(dpt)/(ppi)
(dt) /(ppi)
(Height of Notch)/(Systolic peak)
(hnt)/(Height of Notch)
(dpt)/(Diastolic peak)
(Systolic peak)/(pi-spt)
(Systolic peak)/(pi-hnt)
Waveform width at 25% amplitude of systolic peak (Width 25%)
Waveform width at 50% amplitude of systolic peak (Width 50%)
Waveform width at 75% amplitude of systolic peak (Width 75%)
Diastolic width at 25% amplitude of systolic peak (w25_sdt)
Diastolic width at 50% amplitude of systolic peak (w50_sdt)
Diastolic width at 75% amplitude of systolic peak (w75_sdt)
Diastolic width at 90% amplitude of systolic peak (w90_sdt)
Width(25%)IR/Roundness of systolic peakG
h25_sdtR/h25_sdtIR
h25_sdtR/h50_sdtIR
h50_sdtG/h25_sdtIR
Diastolic_peakR/Diastolic_peakIR
Diastolic_peakR/Diastolic_peakG
Diastolic_peakIR/Diastolic_peakG
Diastolic_peakIR/Diastolic_peakR
Roundness of systolic peak (Waveform AUC at 75% of systolic amplitude)
(Width 25%)/ spt
(Width 25%)/(hnt)
(Width 25%)/(dpt)
(Width 25%)/(dt)
(Width 25%)/(pi)
(Width 50%)/(spt)
(Width 50%)/(hnt)
(Width 50%)/(dpt)
(Width 50%)/(dt)
(Width 50%)/(pi)
(Width 75%)/(spt)
(Width 75%)/(hnt)
(Width 75%)/(dpt)
(Width 75%)/(dt)
(Width 75%)/(pi)
First max of first derivative (a_fir)
Position first max of first derivative (t_a_fir)
First max of second derivative (a_sec)
Position first max of second derivative(t_a_sec)
First minimum peak first derivative of the PPG waveform after a1 (b_fir)
Interval from foot of PPG waveform to b_fir (tb_fir)
First minimum peak first derivative of the PPG waveform after a1 (b_sec)
Interval from foot of PPG waveform to b_sec (tb_sec)
(b_sec)/(a_sec)
(b_fir)/(a_fir)
(ta_fir)/(ppi)
(tb_fir)/(tpp)
(ta_sec)/(ppi)
(tb_sec)/(ppi)
(ta_fir-ta_sec)/(ppi)
(tb_fir-tb_sec)/(ppi)
(Height)/(dt)
(Weight)/(dt)
(BMI)/(dt)
(Height)/(spt)
(Weight)/(spt)
(BMI)/(spt)
(Height)/(hnt)
(Weight)/(hnt)
(BMI)/(hnt)
(Height)/(dpt)
(Weight)/(dpt)
(BMI)/(dpt)
(Height)/(pi)
(Weight)/(pi)
(BMI)/(pi)
(Height)/(ppi)
(Weight)/(ppi)
(BMI)/(pi)
Mean
Median
Standard Deviation
Percentile
Mean Absolute Deviation
Inter Quartile Range
Skewness
Kurtosis
Shannon Entropy
Amplitude first peak Fast Fourier Transform PPG signal (P1)
Amplitude second peak Fast Fourier Transform PPG signal (P2)
Frequency first peak Fast Fourier Transform PPG signal (F1)
Frequency second peak Fast Fourier Transform PPG signal (F2)
Area under the curve from 0 to 2 Hz fast Fourier transform PPG signal (AUC0-2)
Area under the curve from 2 to 5 Hz fast Fourier transform PPG signal (AUC2-5)
(AUC0-2)/(AUC2-5)
(P1)/(P2)
(F1)/(F2)
Maximum Frequency in signal spectrum
Magnitude at maximum frequency
Spectral Entropy

1. A method for processing a photoplethysmography signal, the method comprising the steps of:
emitting light from a wearable device at a plurality of light-wavelengths for measuring a blood pressure of a wearer of the wearable device for at least one light-wavelength;
creating a photoplethysmography segment by acquiring the photoplethysmography signal within a predefined time window, wherein the photoplethysmography signal comprises a plurality of pulses and wherein a pulse is the photoplethysmography signal between two consecutive valleys;
collecting at least one biologically vital sign, such as a heart rate of the wearer, a respiration rate of the wearer, or an oxygen saturation rate of the wearer;
collecting at least one waveform-based parameter proportional to a peak amplitude of the photoplethysmography segment;
collecting at least one time-based parameter proportional to a peak time of the photoplethysmography signal or proportional to a peak width of the photoplethysmography segment;
creating a cardiovascular profile vector of the wearer;
calculating a mean squared error between components of the collected profile vector and components of a profile vector belonging to previously predicted cluster of profile vectors; and
estimating a blood pressure of the wearer using the photoplethysmography segments comprised in the cardiovascular profile of the wearer when the mean squared error is smaller than a first threshold value.

2. The method according to claim 1, further comprising filtering the photoplethysmography segment with the wearable device and using the filtered photoplethysmography segment in the remaining steps of the method.

3. The method according to claim 1, wherein creating a cardiovascular profile vector of the wearer comprises concatenating photoplethysmography segments with at least one of:
the collected at least one waveform-based parameter of the wearer;
the collected at least one time-based parameter of the wearer; and
the collected at least one biologically vital sign of the wearer.

4. The method according to claim 1, further comprising normalizing the collected at least one time-based parameter by multiplying the parameter by a factor inversely proportional to a peak width of the photoplethysmography segment.

5. The method according to claim 1, further comprising emitting light from the wearable device comprising light-wavelengths between about 400 nm and about 1000 nm.

6. The method according to claim 1, further comprising emitting light from the wearable device comprising:
a light-wavelength of about 525 nm;
a light-wavelength of about 660 nm; and
a light-wavelength of about 880 nm.

7. The method according to claim 1, wherein, for at least one photoplethysmography segment, the wearable device is configured for:
calculating at least one of the following values for each pulse of the photoplethysmography segment:
a pulse wave amplitude left by calculating a difference between amplitudes of a first peak and a first valley of the pulse;
a pulse wave amplitude right by calculating a difference between amplitudes of the first peak and a second valley of the pulse;
a pulse wave duration by calculating a difference between a time of the second valley and a time of the first valley of the pulse;
a rise time by calculating a difference between a time of the first peak and a time of the first valley of the pulse; and
a systolic-to-diastolic duration ratio by calculating the ratio between a difference between the rise time and a difference between the time of the second valley and the time of the first peak of the pulse; and
eliminating a photoplethysmography pulse when:
the rise time of the pulse is outside a predefined first range; or
the pulse wave duration of the pulse is outside a predefined second range; or
a ratio or an inverse ratio between the pulse wave amplitude right and the pulse wave amplitude left is smaller than a second threshold value; or
the systolic-to-diastolic duration ration is larger than a predefined third threshold value; and
calculating a signal quality index of the photoplethysmography segment by calculating a ratio of non-eliminated photoplethysmography pulses over a total number of photoplethysmography pulses comprised in the photoplethysmography segment.

8. The method according to claim 7, further comprising eliminating the photoplethysmography segment when signal quality index of the photoplethysmography signal is lower than a fourth threshold value.

9. The method according to claim 8, further comprising setting the fourth threshold value at 20%.

10. The method according to claim 1, further comprising:
collecting demographic information of the wearer, such as age, sex, body weight and height; and
adding the demographic information into the cardiovascular profile vector of the wearer.

11. The method according to claim 1, further comprising grouping profile vectors of a plurality of wearers into a plurality of clusters based on a similarity in at least one of the biologically vital signs and/or based on a similarity in the demographic information of the wearers by a using a silhouette coefficient to determine an optimal number of clusters for establishing a K-Means clustering on the profile vectors.

12. The method according claim 11, further comprising training a Random Forest model on each cluster of profile vectors by combining a plurality of decision trees and training each decision tree on a separate subset data of the cluster.

13. The method according to claim 11, further comprising:
assigning a new wearer to one of the plurality of clusters based on at least one of the biologically vital signs of the new wearer and/or based on the demographic information of the new wearer; and
using the Random Forest model corresponding to the cluster as a starting point, and fine-tuning the starting point to create a cardiovascular profile for the new wearer using a regularization framework such as a Lasso regression, a Ridge regression or an Elastic Net.

14. The method according to claim 1, further comprising updating the cardiovascular profile of the wearer by performing the steps of claim 1 on new sets of acquired photoplethysmography segments.

15. The method according to claim 1, further comprising emitting light onto a skin of a wearer and receiving light emitted from the skin of the wearer.

16. The method according to claim 2, further comprising filtering the photoplethysmography segment with a bandpass filter.

17. The method according to claim 1, further comprising performing the steps of claim 1 with a computer system.
